(19) United States
(12) Patent Application Publication
(10) Pub. No.: US 2005/0222556 A1
(43) Pub. Date: Oct. 6, 2005

(54) Apparatus and method for hyperthermia treatment

(75) Inventors: Shigeki Ariura, Ebina-shi (JP); Seiichi Ootsubo, Hadano City (JP); Akira Sakaguchi, Minami-ashigara City (JP); Norihiko Hareyama, Hino-shi (JP)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignees: TERUMO KABUSHIKI KAISHA, Tokyo (JP); OLYMPUS CORPORATION, Tokyo (JP)

(21) Appl. No.: 11/092,561

(22) Filed: Mar. 29, 2005

(30) Foreign Application Priority Data
Mar. 31, 2004 (JP) 2004-104978

(51) Int. Cl. A61B 18/20

(52) U.S. Cl. 606/12; 606/16

(57) ABSTRACT

The apparatus and method of the invention is equipped with an applicator for heating a target are of body tissue by a heating unit, a temperature sensor for measuring the body tissue's temperature, a target temperature calculating unit for calculating target temperatures based on temperature change pattern set up from an operating unit, and a control unit for controlling the laser power from the heating unit so that the temperature measured by the temperature sensor follow the calculated temperature change pattern.

### BACKGROUND OF THE INVENTION

1. Field of the Invention

The present invention relates to an apparatus and method for hyperthermia treatment intended for treating living body tissue including lesions by irradiating living body tissue with energy.

2. Description of the Related Art

Apparatuses for hyperthermia treatment intended for treating affected regions of body tissue, degenerating and coagulating the affected regions by heating the regions by means of irradiation mainly with electromagnetic energies such as laser, have been known. A typical apparatus for hyperthermia treatment provides treatments by irradiating tissue with electromagnetic waves from an emitting unit, inserting the energy emitting unit into body lumens or hollow organs such as blood vessels, digestive tube, urinary tract, abdominal cavity, and thoracic cavity, or pressing it against the surface of body tissue such as skins and internal organs.

In a typical treatment using such an apparatus for hyperthermia treatment, it is necessary to apply heat for a certain period of time at a specified temperature in order to cause degeneration and coagulation of body tissue containing a lesion area. On the other hand, however, it is a common practice to set the irradiation time to a very minimum in order to raise the temperature to a level required for degeneration and coagulation for minimizing the effect on normal tissue.

In order to accomplish it, an apparatus for hyperthermia treatment of the related art disclosed by the Unexamined Publication 2002-102268 (A), for example, controls the energy irradiation intensity and the irradiation time to reach the set temperature as quickly as possible and stabilize the temperature to the set level efficiently by radiating electromagnetic wave based on the measurement of body tissue temperature.

However, in case of apparatuses for hyperthermia treatment of the related art using laser as the source of electromagnetic energy, the more they try to shorten the time required for reaching the temperature required for degeneration and coagulation, the shallower the highest temperature point becomes in body tissue, as the rate of laser dispersion increases with the degeneration and coagulation of the body tissue. Consequently, it had a problem of narrowing the range of reaching the degeneration and coagulation, making treatments in the depth direction more difficult.

### SUMMARY OF THE INVENTION

The present invention was made to improve such problems and its object is to provide an apparatus for hyperthermia treatment to treat only a targeted body tissue area. More specifically, it is intended to provide an apparatus for hyperthermia treatment for treating areas of wider depth ranges of body tissue.

The apparatus for hyperthermia treatment according to the present invention has: a heating unit for heating a target body tissue area; a temperature measuring unit for measuring the temperature of said body tissue including said target area; a target temperature setup unit for setting up a temperature change pattern to be generated by said heating unit; and a control unit for controlling said heating unit so that the temperature measured by said temperature measuring unit conforms with said temperature change pattern.

The apparatus for hyperthermia treatment according to the present invention preferably has a temperature change pattern such that the differential coefficient obtained by differentiating temperature by time increases with the elapsed time. This enables us to provide hyperthermia for treating only a certain deep body tissue area in a stable manner.

A method for hyperthermia treatment according to the present invention has: setting a temperature change pattern; measuring a temperature of a body tissue including a target area; and heating the target area so that the measured temperature changes in accordance with said temperature change pattern.

The objects, features, and characteristics of this invention other than those set forth above will become apparent from the description given herein below with reference to preferred embodiments illustrated in the accompanying drawings.

### BRIEF DESCRIPTION OF THE DRAWINGS

FIG. 1 shows the constitution of an apparatus for hyperthermia treatment according to an embodiment of the present invention;

FIG. 2 is a side view of an applicator:

FIG. 3 is a cross section showing the constitution of an insertion unit located at a distal end of the applicator;

FIG. 4 is a block diagram showing the function of a controller;

FIG. 5 shows graphs of target temperature changes during hyperthermia treatments;

FIG. 6 shows diagrams for describing various output control patterns during a temperature rising period;

FIG. 7 is an explanatory diagram for this temperature control summarizing the relations between the measured temperature vs. the calculated temperature and the calculated next target temperature vs. the change rate of measured temperature from the last measurement;

FIG. 8 shows diagrams for describing various output control patterns during a temperature equilibrium period;

FIG. 9 is an explanatory diagram for this temperature control summarizing the relations between the measured temperature vs. the set temperature and the change rate of measured temperature since the last measurement;

FIG. 10 is a flow chart showing a temperature control process sequence;

FIG. 11 is a flow chart showing a temperature control process sequence;

FIG. 12 is a diagram showing an example case of temperature control:

FIG. 13 is a flow chart showing a temperature control process sequence in a second embodiment;

FIG. 14 is a graph showing a target temperature change pattern in a third embodiment;

FIG. 15 is a functional block diagram of a controller in a fourth embodiment;

FIG. 16 is a flow chart showing a temperature control process sequence in a fourth embodiment;

FIG. 17 is a lateral cross section showing the structure of a deep area temperature sensor used for temperature measurements of deep body tissue areas;

FIG. 18 shows schematic views of an insertion passage of the deep area temperature sensor;

FIG. 19 shows cross sections indicating an insertion status of an applicator and a deep area temperature sensor located in a body tissue;

FIG. 20 shows cross sections indicating an insertion status of an applicator and a deep area temperature sensor located in body tissue;

FIG. 21 is a graph showing temperature measurement values on a surface of a body tissue sample relative to laser irradiation time;

FIG. 22 is a graph showing temperature measurement values in a deep body tissue area relative to laser irradiation time;

FIG. 23 is a model cross-sectional diagram of a body tissue sample after laser irradiation; and

FIG. 24 shows diagrams for describing thermal degeneration of body tissue due to laser irradiation.

### DETAILED DESCRIPTION OF THE PREFERRED EMBODIMENT

The embodiments of this invention will be described below with reference to the accompanying drawings.

### First Embodiment

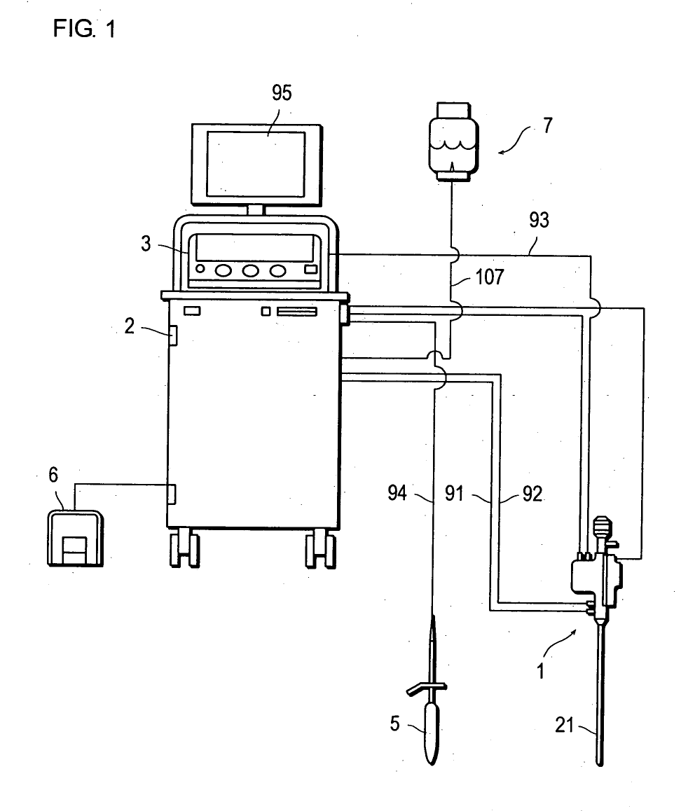

FIG. 1 shows the constitution of an apparatus for hyperthermia treatment according to an embodiment of the present invention.

The apparatus for hyperthermia treatment has an applicator 1, a controller 2, a laser generator 3, a rectal probe 5, a foot switch 6, and a cooling water refilling tank 7.

The applicator 1, the laser generator 3, the rectal probe 5, the foot switch 6, and the cooling water refilling tank 7 are all connected to the controller 2.

A cooling device (not shown) for supplying cooling water as cooling fluid to the applicator 1 is provided inside the controller 2. The controller consists of a cooling water pack for holding an approximately constant amount of cooling water, a pump (fluid feeder) for circulating the cooling water, and a Peltier effect unit for maintaining the cooling water at a predetermined temperature. The cooling water pack cools the water temperature to a fixed temperature by being pressed against the Peltier effect unit.

The applicator 1 is an emitting unit and radiates laser sideway to irradiate body tissue. The applicator 1 is connected with an optical fiber 93 used for transmitting laser generated in the laser generator 3 to the emitting portion of an insertion unit 21. Therefore, the applicator 1 and the laser generator 3 including the optical fiber 93 function together as a heating unit.

In applying hyperthermia treatment to a body tissue, this slender-shaped insertion unit 21 is inserted into the body tissue such as the urinary tract to irradiate laser from the emitting unit provided on the insertion unit 21. The apparatus for hyperthermia treatment treats lesions such as benign prostatic hyperplasia and various cancers and tumors.

The applicator 1 applies heat only a target area, a lesion residing deep in a vital tissue of a living body, while cooling the insertion unit 21 to suppress the heating of the laser radiating surface thus to avoid heating of normal body tissue by the laser radiating surface. For this purpose, the applicator 1 is connected with a supply passage 91 and a discharge passage 92 to circulate the cooling water from the cooling device provided inside the controller 2. Therefore, the applicator 1 cools not only the insertion unit 21 but also areas of body tissue in contact with the insertion unit 21 by this cooling water.

The controller 2 controls the entire operation of the apparatus for hyperthermia treatment by signals detected by various sensors and micro switches provided in the applicator 1 and the rectal probe 5, as well as ON/OFF signals issued by the footswitch 6.

A user interface 95 is provided at the top of the controller 2 for displaying specific information to the operator as well as for accepting various settings and operations. The laser interface 95 is a touch-screen type operating panel including a display screen.

The laser generator 3 emits laser output controlled by instructions from the controller 2, which controls output conditions such as laser power values, laser pulse times, laser pulse rates, and laser radiation times. It is also possible to set the abovementioned output conditions arbitrarily by switches and dials on the laser generator 3.

The rectal probe 5 is inserted into the rectum through the anus and detects the temperature of the rectal wall located adjacent to the prostate gland in order to watch abnormal heating of the rectum during the treatment. The rectal probe 5 is equipped with a plurality of temperature sensors and their detection values are transmitted to the controller 2 via sensor signal leads 94.

The foot switch 6 sends ON/OFF signals for prompting the controller 2 to radiate laser when it is stepped on by the operator. When the laser irradiation preparation procedure is completed and the foot switch 6 is stepped on, the laser generator 3 radiates laser. However, while the normal termination of the laser output is conducted by an instruction from the controller, the laser output can be turned OFF arbitrarily by operating the foot switch in addition to the normal termination operation.

The cooling water refilling tank 7 is a container for holding cooling water to be used for refilling the cooling device built into the controller 2. The cooling water refilling tank 7 and the cooling device are connected by a cooling water refilling passage 107.

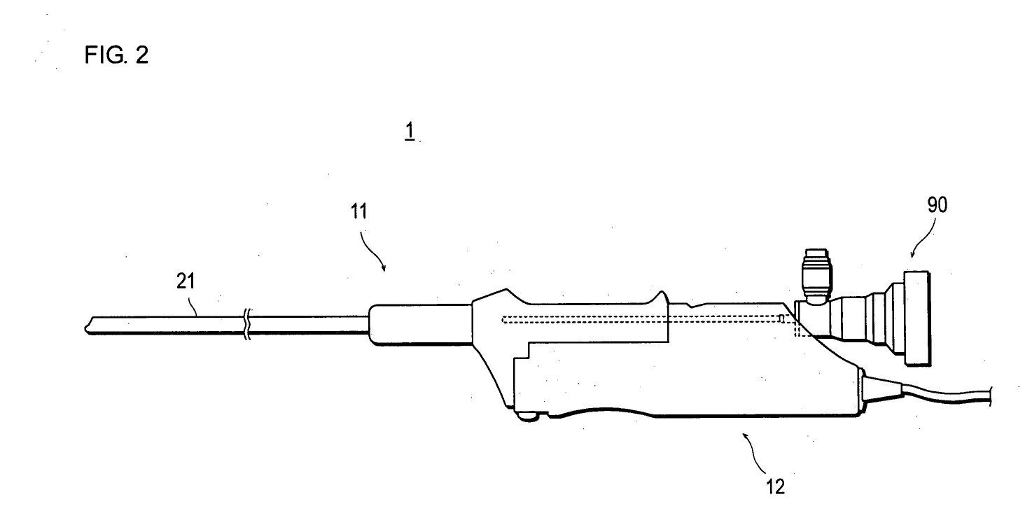

FIG. 2 is a side view of an applicator 1.

The applicator 1 is preliminarily sterilized in advance consisting of a disposable main unit 11, which can be disposed of after use, and a drive unit 12 which can be used repeatedly.

The insertion unit 21 is provided at the distal end of the main unit 11. The main unit 11 and the drive unit 12 are interchangeable and removable. Fastening by means of fastening members or fitting by means of fingers and other devices can be used for connecting the main unit 11 with the drive unit 12.

The drive unit 12 serves as a unit for electrically driving the device and contains a mirror motor (not shown) by providing reciprocating motions of the mirror to be described later.

The insertion unit 21 has an endoscope 90 built into it in order to facilitate observation of internal organisms through the insertion unit 21. The endoscope 90 has a suitable field of vision to obtain a field of observation through a window provided at the front portion of the insertion unit 21. The endoscope 90 is equipped with an optical fiber bundle, a protection tube, and a focusing lens provided at the distal end thereof. A camera head is provided at the proximal end of the endoscope 90 in order to feed back images.

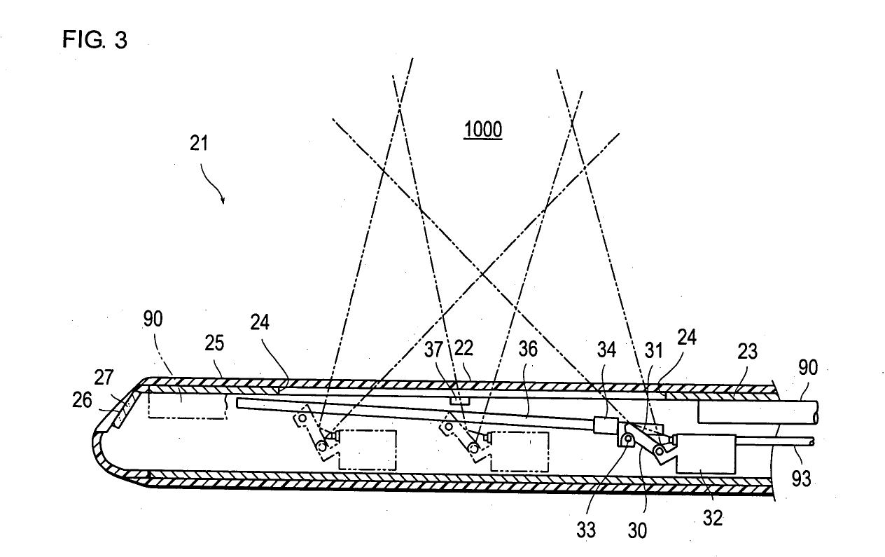

FIG. 3 is a cross section showing the constitution of the insertion unit 21 located at the distal end of the applicator.

As shown in FIG. 3, the insertion unit 21 is equipped with a side window 22 extending in the longitudinal direction and an emitting unit 30, which emits laser in the direction of the side window 22 and is reciprocally movable, is provided inside the insertion unit 21. The insertion unit 21 is equipped with a long inner layer pipe 23, and the emitting unit 30 has a flat reflecting surface (mirror) 31 for reflecting laser.

The inner layer pipe 23 of the insertion unit 21 consists of a tube-like member made of a hard material such as stainless steel. An opening 24 is formed at a distal end of the inner layer pipe 23 for allowing laser to be transmitted. The outer circumference of the inner layer pipe 23 including the opening 24 is covered by an outer layer tube 25 of excellent laser transmissibility. The opening 24 covered by the outer layer tube 25 constitutes a side window 22.

A front window 26 is provided at a distal end of the insertion unit 21 for frontal observation when the insertion unit 21 is inserted into a living body. The frontal window 26 is provided with a light transmitting plate 27 which transmits light and this light transmitting plate 27 is fitted into said window's frame to be affixed.

An optical fiber 93 is provided inside the insertion unit 21 for transmitting laser. A drive output is transmitted from the mirror motor to the optical fiber 93 to cause it to reciprocate along the axial direction of the insertion unit 21. The optical fiber 93 is covered by a protective pipe inside the insertion unit 21 except the distal end in order to prevent it from damage and bending. A fixture 32, to which the emitting unit 30 is mounted rotatably, is affixed to the optical fiber 93 in the vicinity of the distal end thereof. The fixture 32 slides in parallel with the axis of the insertion unit 21 in accordance with the reciprocating movement of the optical fiber 93.

A temperature sensor 37 is provided inside the insertion unit 21 for measuring the surface temperature of body tissue irradiated with laser, for example, the urethra surface temperature. The temperature sensor 37 is located approximately in the middle of the lengthwise direction of the side window 22 so that it does not interfere with the movement of the emitting unit 30 or with the laser irradiation, and is located at a position where it always has an entire view of the specific body tissue through the side window 22. The reason for locating it at this position is that it is the position where a highest temperature is likely to be reached by laser irradiation and it allows the measurement of the surface temperature of the laser irradiated area in such a way as to have correlation with the deep body tissue temperature. The temperature sensor 37 can be a thermister, thermocouple, infrared sensor, etc.

A protrusion 33 is provided on each side of the distal end of the emitting unit 30. A slider 34 that rotatably supports the protrusions 33 is slidably supported by a pair of slider guides 36 provided inside the insertion unit 21. The slider guide 36 is tilted against the axial direction of the insertion unit 21. Therefore, the emitting unit 30 reciprocates due to the action of the slider guide 36 in accordance with the reciprocating motion of the optical fiber 93. Thus, the slider 34 and the slider guide 36 constitute an angle changing unit for changing the angle of the reflecting surface 31 in coordination with the motion of the emitting unit 30. This makes sure that the laser beam radiating from the optical fiber 93 will be directed toward the targeted area. The locations of the emitting unit 30 and the fixture 32 shown in solid lines in FIG. 3 are their rear end positions.

A lumen for cold water supply and a lumen for drain (both not shown) are formed in the inside of the insertion unit 21. Cold water is used for cooling the surface of the body tissue being irradiated by laser and the emitting unit 30. The lumen for cold water supply is connected to the supply passage 91 and the drain lumen is connected to the drain passage 92 (see FIG. 1). The cold water supplied via the supply passage 91 flows into the supply lumen to be sent to the vicinity of the distal end of the insertion unit 21 and returns to the cooling device via the drain passage 92.

The cooling efficiency is improved by circulating the cooling water inside the insertion unit 21. The cooling water temperature is not specifically limited as long as it can attenuate damages of the emitting unit 30 and the irradiated surface of body tissue by laser irradiation, but it is preferable to be maintained in the range of 0 to 37° C., or more preferably 8 to 25° C., which provide a lower probability of cold injury and a higher cooling efficiency. It is preferable to use sterilized liquid, for example, sterilized pure water or sterilized physiological saline as cooling water.

The emitting unit 30 rises up to a posture approximately perpendicular to the axial direction of the insertion unit 21 when it is positioned at the distal end position (shown by phantom lines on the left side of FIG. 3) and reflects laser at a small reflection angle. The emitting unit 30 tilts to a posture approximately parallel to the axial direction of the insertion unit 21 when it is positioned at the proximal end position (shown by solid lines in FIG. 3) and reflects laser at a large reflection angle. Therefore, as the emitting unit 30 reciprocates while changing its tilt angle constantly thus moving the radiating position of laser radiated through the side window 22, the laser's optical axis is always on a target point within a target area 1000, which is a heating area. In other words, the laser irradiates constantly only the target point, while irradiating other body tissues such as the surface layer. Thus, the target point is heated by irradiating laser and reaches the desired temperature. On the other hand, other body tissues such as the surface layer are heated only slightly as they receive laser only short periods of time. The reciprocation of the emitting unit 30 is preferably adjusted in such a way as to achieve a constant travel speed.

During a hyperthermia treatment, the emitting unit 30 reciprocates in the axial direction at a frequency of 0.1 to 10 Hz, preferably 3 to 6 Hz. The laser irradiating body tissue can be either a divergent, parallel or convergent light. The laser used in this invention can be any kind as long as it has a deep-reaching capability against body tissue. However, laser wavelengths in the range of 750 to 1300 nm or 1600 to 1800 nm provide the best deep-reaching capability and therefore are preferable. The laser generator 3 that generates laser having wavelengths in said range include devices using gaseous laser such as He—Ne laser, solid laser such as Nd-YAG laser, and semiconductor laser such as GaAlAs laser.

The outer diameter of the insertion unit 21 is not limited as long as it allows insertion into body cavities. However, the outer diameter of the insertion unit 21 is preferably 2 to 20 mm, or more preferably 3 to 8 mm.

Next, laser irradiation control will be described below:

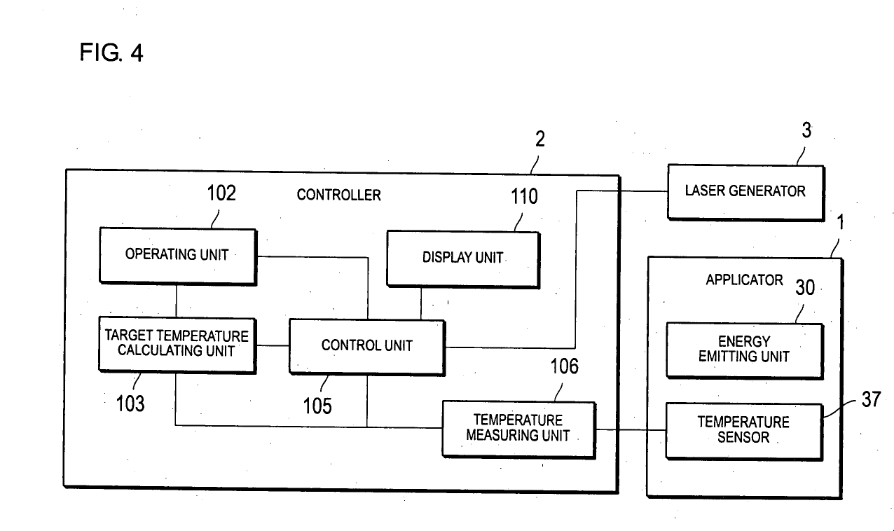

FIG. 4 is a block diagram showing the functions of the controller 2.

The controller 2 has: an operating unit 102 for the user to do various settings and operations including the temperature chance pattern; a target temperature calculating unit 103 for calculating a target temperature change pattern based on a heating temperature and time set up in the operating unit 102; a display unit 110 for displaying energy value to be actually outputted; a temperature measuring unit 106 for measuring the heating area temperature; and a control unit 105 for controlling the laser output.

The operating unit 102 is the user interface 95 shown in FIG. 1, and is used by the operator for setting the temperature change pattern required by for treatment and various other setups using a touch panel type input device.

The target temperature calculating unit 103 is used for calculating the target temperature as the time goes during a treatment based on the heating temperature, the heating time and the target temperature change pattern set up from the operating unit 102. Therefore, the target temperature calculating unit 103 functions as a target temperature setting unit in coordination with the operating unit 102.

The control unit 105 is used for controlling various units of the controller and the power of the laser generator, so that it functions not only as a control unit but also as a temperature change rate calculating unit and a target change rate calculating unit.

The temperature measuring unit 106 is connected with the temperature sensor 37 in the applicator 1 to measure the surface temperature of the heated area.

The energy value indicated by the display unit 110 is displayed on the user interface 95 of the controller 2. The energy value itself can be displayed numerically or an indicator expressed in a ratio against the maximum power.

This controller is physically a computer equipped with a CPU, a memory, a hard disk, a display, and other interface, and executes a program prepared according to the sequence to be described later to control laser irradiation along a target temperature change pattern.

Next, temperature control in hyperthermia treatment will be described below.

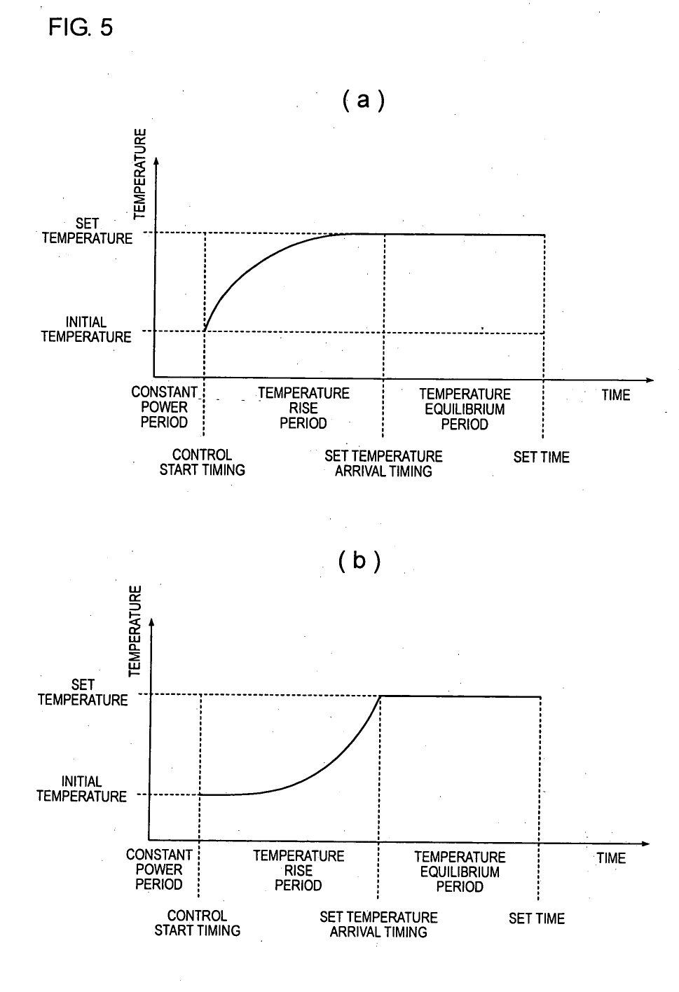

FIG. 5 is a graph showing a target temperature change pattern during a typical hyperthermia treatment.

Such a target temperature change pattern is set up in order to raise temperature along this target temperature change pattern during a hyperthermia treatment.

The target temperature change pattern mentioned here consists of a constant power period, a temperature rise period, and a temperature equilibrium period. In other words, the target temperature change pattern is a pattern with a plurality of change rates.

During the constant power period, the energy value is constant regardless of the temperature. In the temperature rise period, the energy value is controlled in such a way that the temperature rises in accordance with the temperature rise curve that defines the desired temperature rise curve up to a point when it reaches the set temperature. In the temperature equilibrium period, the energy value is controlled to maintain the set temperature.

The constant power period continues for a predetermined length of time of 1 to 30 seconds, and the energy value during this period is set to a relatively lower value within the power adjustment range. This is so that the laser power control is executed only after the conditions such as energy value and cooling water are stabilized. This makes it possible to provide a more stable control.

The temperature rise period is defined as a period by the initial temperature when the control starts after the constant power period is over, the temperature set by the user, and the time for the temperature rising period spanning from the initial temperature to the set temperature. The temperature target expressed by such a curve as indicated in the drawing is called a temperature rise curve. The temperature rise time of this period can be set to a predetermined time period (approximately 30 to 180 seconds) or a predetermined ratio to the setup time period. The predetermined ratio is defined, for example, as “two-thirds of (setup time−constant power time).”

Two different examples of temperature rise curve are shown here as FIG. 5(a) and FIG. 5(b). Example (a) shows a type where the temperature rises fast in the first half of the period and slowly in the second half, while example (b) is a type where the temperature rises slowly in the first half of the period and fast in the second half. As to the difference in temperature rise in body tissue between these temperature rise curves, it will be described later referring to actual examples.

The temperature rise curve of (a) is defined by the following formula (1):
Temperature=A×(time−temperature rise time)²+setup temperature (1)
where A=(initial temperature−setup temperature)/(temperature rise time)².

The temperature rise curve of (b) is defined by the following formula (2):
Temperature=B×(time)²+initial temperature (2)
where B=(setup temperature−initial temperature)/(temperature rise time)².

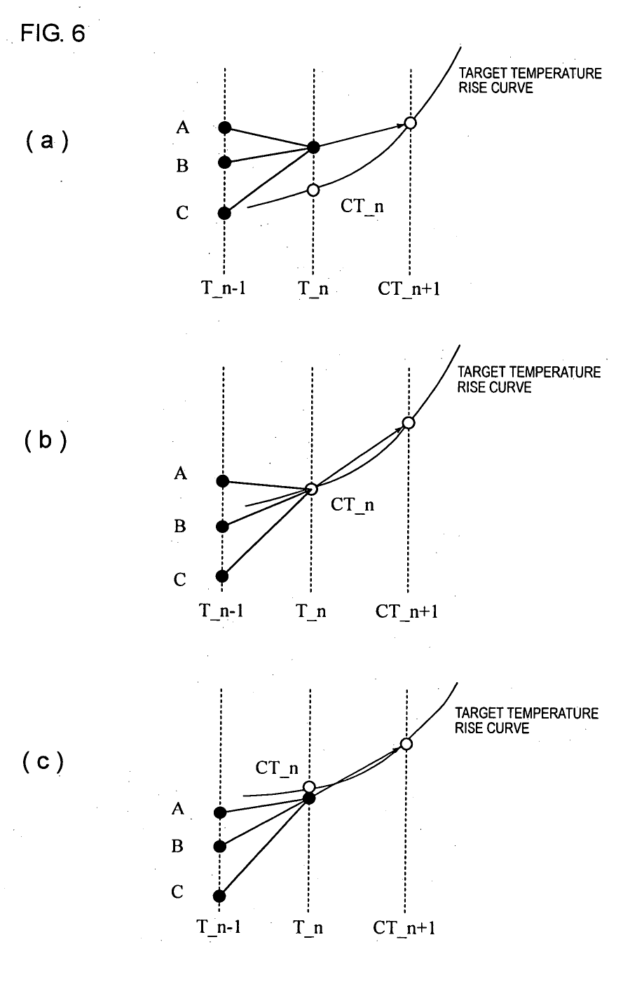

FIG. 6 is an explanatory drawing for describing how the laser power control is executed during the temperature rise period. FIG. 6 shows the case of FIG. 5(b) as an example of the temperature rise curve in the temperature rise period. The laser power control method used in the case of FIG. 5 (a) is the same.

The laser power control is performed using the temperature measured at present time T_n, the temperature measured during last control period T_n−1, the target temperature to be held at preset time calculated from the temperature rise curve CT_n, and the target temperature for the next control period CT_n+1. The measured temperature is the temperature measured on the surface of the laser irradiated surface using the temperature sensor 37 (same hereinafter).

FIG. 6(a) is a case where the measured temperature T_n is higher than the calculated temperature CT_n. At this point, the change rate CT_n+1−T_n, i.e., the change required from the temperature measured at present time T_n to the next target calculated temperature CT_n+1, is calculated and compared with the change rate of measured temperature since the last measurement, i.e., T_n−T_n−1. If the change rate is equal (FIG. 6(a)-B), or the change rate of measured temperature, i.e., T_n−T_n−1, is smaller (FIG. 6(a)-A), it is judged that the temperature is nearing the temperature rise curve and the laser power is not changed. The laser power is lowered only if the change rate of measured temperature, i.e., T_n−T_n−1, is larger (FIG. 6(a)-C).

FIG. 6(b) is a case where the measured temperature T_n is equal to the calculated temperature CT n. The output is not changed in this case.

FIG. 6(c) is a case where the measured temperature T_n is lower than the calculated temperature CT_n. At this point, the change rate CT_n+1−T_n, i.e., the change required from the temperature measured at present to the next target calculated temperature, i.e., CT_n+1, is calculated and compared with the change rate of measured temperature since the last measurement, i.e., T_n−T_n−1. If the change rate is equal (FIG. 6(c)-B), or the change rate of measured temperature, i.e., T_n−T_n−1, is larger (FIG. 6(c)-C), it is judged that the temperature is nearing the temperature rise curve and the output is not changed. The laser power is increased only if the change rate of measured temperature, i.e., T_n−T_n−1, is smaller (FIG. 6(c)-A).

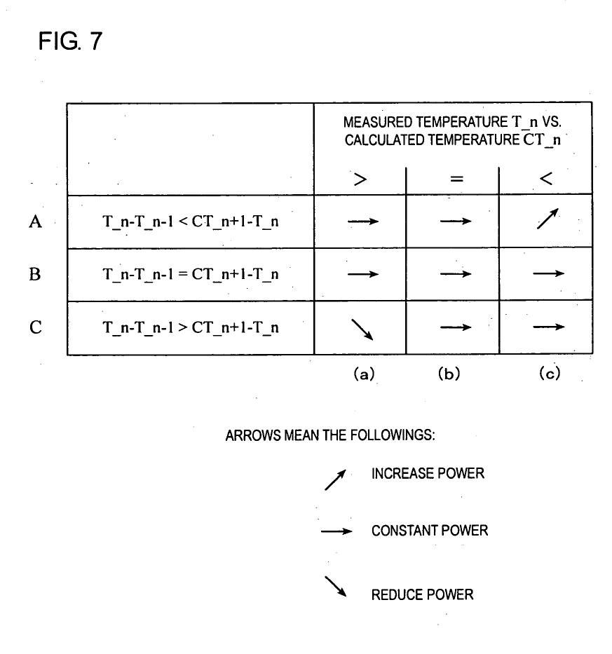

FIG. 7 is an explanatory diagram for this temperature control summarizing the relations between the measured temperature T_n vs. the calculated temperature CT_n and the calculated next target temperature vs. the change rate of measured temperature since the last measurement. The combination of the items (a), (b), (c), A, B, and C in FIG. 6 corresponds to the combination of the items (a), (b), (c), A, B, and C in FIG. 7 respectively.

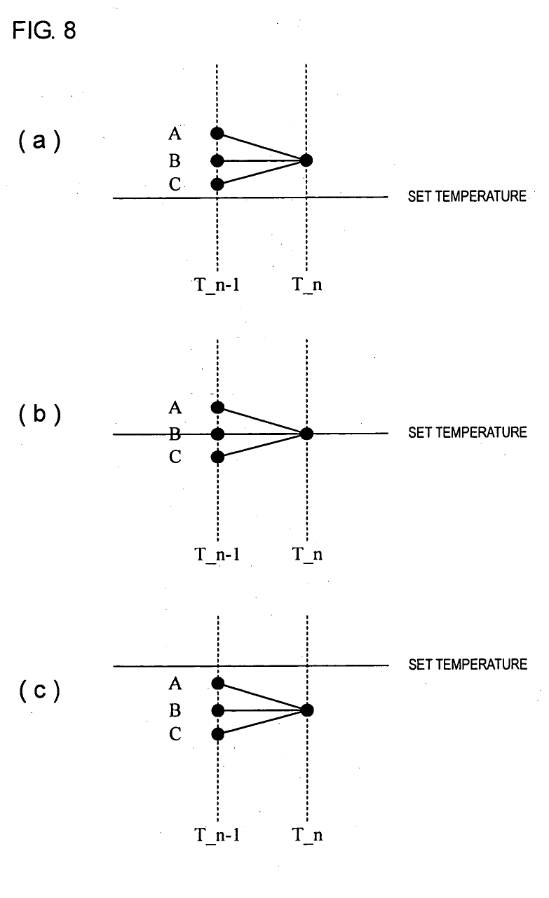

FIG. 8 is an explanatory drawing for describing how the laser power control is executed during the temperature equilibrium period. The laser power control during this period can be performed using the temperature measured at present time, T_n, and the temperature measured last time, T_n−1.

FIG. 8(a) is a case where the measured temperature T_n is higher than the set temperature. In this case, if the change rate of measured temperature since the last measurement, T_n−T_n−1, is calculated and the change rate is negative (FIG. 8(a)-A), it is judged that it is nearing the set temperature so that the laser power is not changed. If the change rate of measured temperature, T_n−T_n−1, is zero (FIG. 8(a)-B) or positive (FIG. 8(a)-C), the laser power is lowered.

FIG. 8(b) is a case where the measured temperature T_n is equal to the set temperature set_temp. The laser power is not changed in this case.

FIG. 8(c) is a case where the measured temperature T_n is lower than the set temperature. In this case, if the change rate of measured temperature since the last measurement, T_n−T_n−1, is calculated and the change rate is positive (FIG. 8(c)-C), it is judged that it is nearing the specified temperature so that the laser power is not changed. If the change rate of measured temperature, T_n−T_n−1, is zero (FIG. 8(c)-B) or negative (FIG. 8(c)-A), the output is raised.

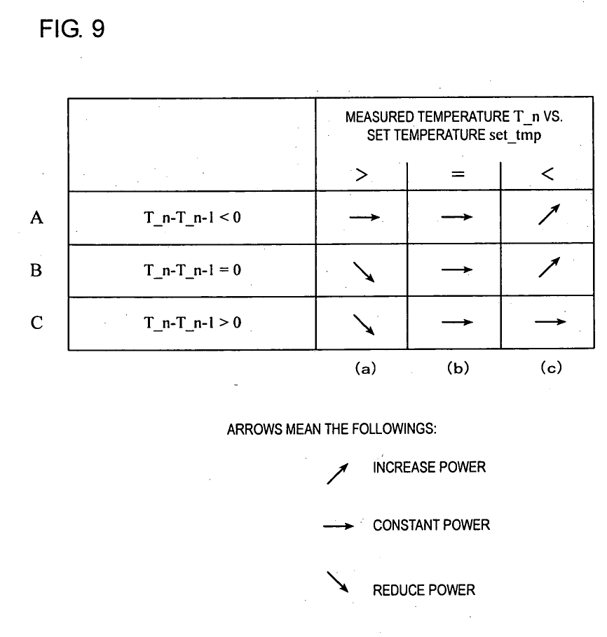

FIG. 9 is an explanatory diagram for this temperature control summarizing the relations between the measured temperature T_n vs. the set temperature set_tmp and the change rate of measured temperature since the last measurement, T_n−T_n−1. The combination of the items (a), (b), (c), A, B, and C in FIG. 9 corresponds to the combination of the items (a), (b), (c), A, B, and C in FIG. 8 respectively.

With such a temperature control, the temperature of the treatment area can be increased along the target temperature change pattern.

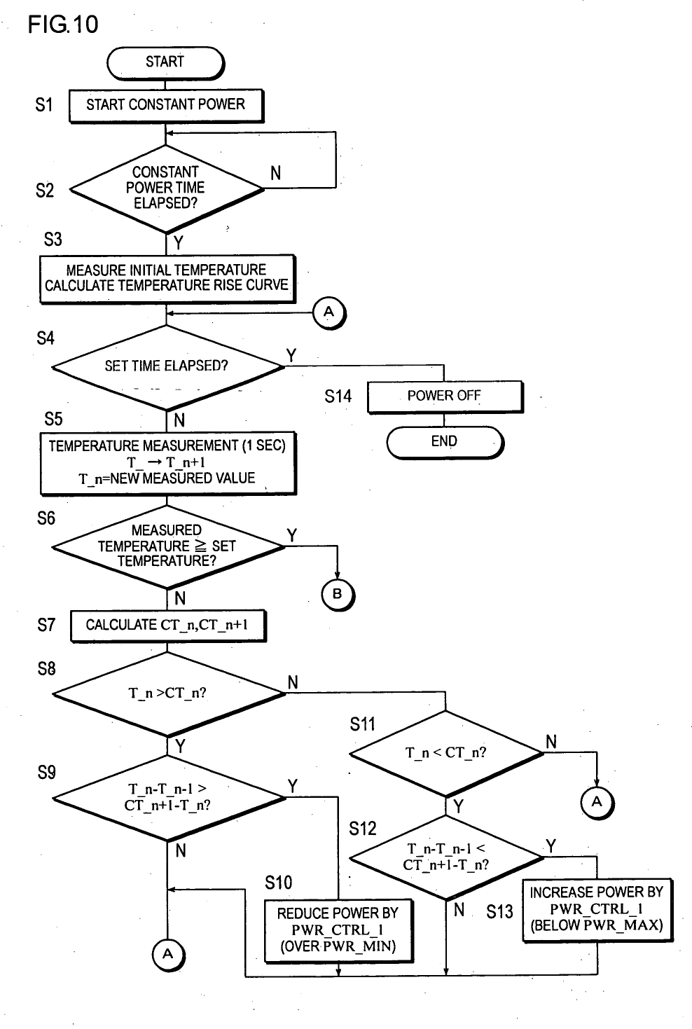

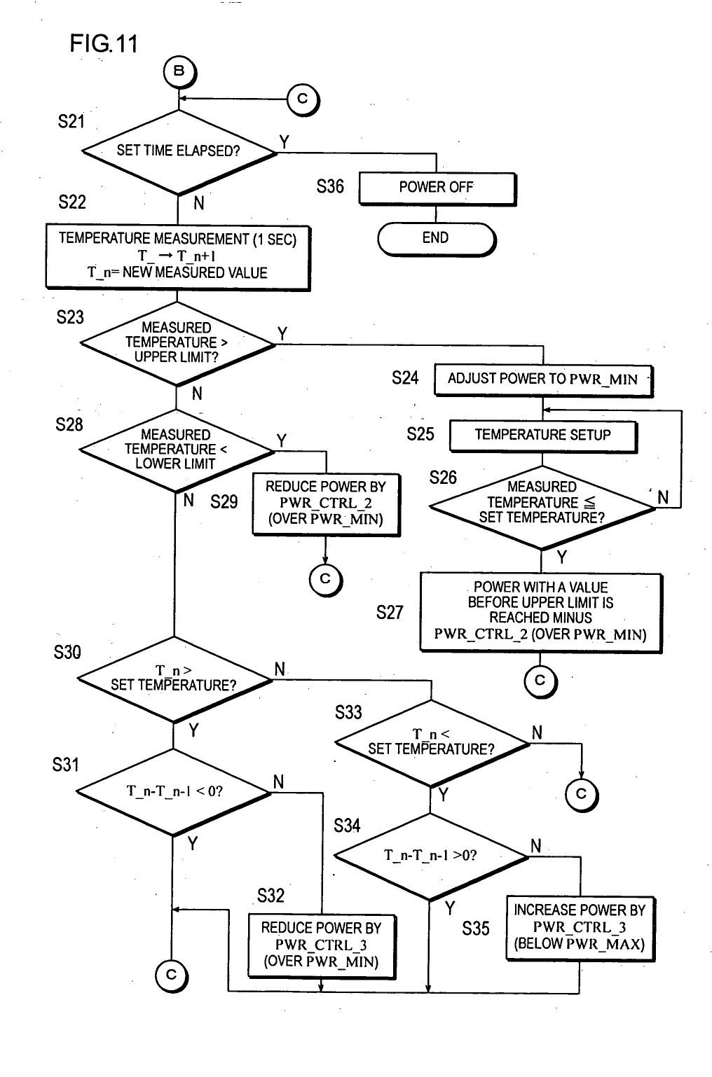

FIG. 10 and FIG. 11 constitute a flow chart showing the temperature control process sequence.

First, the control unit 105 instructs the laser generator 3 to generate a predetermined relatively low power such as 5 W laser power when a treatment starts with the operator's instruction from the operating unit 102 (S1).

Next, the control unit 105 continues to output the laser until a predetermined constant power period is over (S2), and moves to the next step when the constant power period is over.

Next, the control unit 105 stores the temperature measured by the temperature measuring unit 106 at the point when the constant power period is over as the initial temperature, and calculates a temperature rise curve for the temperature rise period (S3). The temperature rise curve is calculated at this time using the aforementioned formula (1) of formula (2). In continuation, the control unit 105 makes a judgment whether the set time has elapsed (S4), stops the laser and terminates the operation (S14) if it has elapsed (S4: Y).

On the other hand, if the set has not elapsed, the control unit 105 instructs the temperature measuring unit 106 to measure the current temperature, and obtains the temperature measurement result (S5). The process of this temperature measurement can be executed, for example, at an interval of 1 second. The value of the last measurement will be stored as T_n−1 and the value of the new measurement as T_n. Next, the control unit 105 makes a judgment as to whether the measured temperature is higher than the set temperature or not (S6) and, if it is higher than the set temperature (S6: Y), it advances to the step S21 (FIG. 11) assuming that the temperature rise period is finished.

On the other hand, if it is not higher than the set temperature, the control unit 105 calculates the temperature which is supposed to be at present time, i.e., CT_n, and the temperature which is supposed to be at the next control time, i.e., CT_n+1 from the temperature rise curve (S7).

Next, the control unit 105 compares the measured temperature, T_n, and the temperature which is supposed to be at present time, CT_n (S8). If the measured temperature is higher (S8: Y) as a result of the comparison, it goes to the step S9, or to the step S11 otherwise.

In the step S9, the control unit 105 compares the change rate CT_n+1−T_n, i.e., the change required from the temperature measured at present T-n to the next target calculated temperature CT_n+1, with the change rate of measured temperature since the last measurement, i.e., T_n−T_n−1. If the change rate of measured temperature T_n−T_n−1 is larger (S9: Y), it instructs the laser generator 3 to lower the power by a predetermined adjustment value PWR_CTRL_{—}1 (e.g., 1 W) (S10). However, it will never be lowered below a predetermined minimum value PWR_MIN (e.g., 1 W). This process corresponds to the pattern C of FIG. 6 (a). On the other hand, if the abovementioned condition is not met in the step S9 (S9: N), it returns to the step S4 without making any output adjustment.

In the step S11, the control unit 105 compares the measured temperature T_n with the temperature which is supposed to be at the present time CT_n. If the measured temperature is lower (S11: Y), it advances to the step S12. Otherwise, it returns to the step S4 (corresponds to FIG. 6(b)).

In the step S12, the control unit 105 compares the change rate CT_n+1−T_n, i.e., the change required from the temperature measured at present T-n to the next target calculated temperature CT_n+1, with the change rate of measured temperature since the last measurement, i.e., T_n−T_n−1. If the change rate of measured temperature T_n−T_n−1 is smaller (S12: Y), it instructs the laser generator 3 to increase the output by a predetermined adjustment value PWR_CTRL_{—}1 (e.g., 1W) (S13). However, it shall not be increased above a predetermined maximum value PWR_MAX (e.g., 30 W). This process corresponds to the pattern A of FIG. 6(c). On the other hand, if the abovementioned condition is not met (S12: N), it returns to the step S4 without making any laser power adjustment.

When the control unit 105 judges in the step S21 that the set time has elapsed (S21: Y), it instructs the laser generator 3 to stop the laser (S36) and terminates all the processes.

On the other hand, if the control unit 105 judges in the step S21 that the set time has not elapsed (S21: N), the control unit 105 instructs the temperature measuring unit 106 to measure the current temperature and obtains the temperature measurement result (S22). The temperature measurement is conducted, for example, at an interval of 1 second. The value of the last measurement will be stored as T_n−1 and the value of the new measurement as T_n.

Next, the control unit 105 makes a judgment as to whether the measured temperature is above an upper limit (S23). The upper limit here is an arbitrary temperature slightly above the set temperature during the temperature equilibrium period in the target temperature change pattern shown in FIG. 5. However, this upper limit changes with the set temperature, and it can be a slightly higher value if the set temperature is low, but it should be a temperature in the range that does not affect body tissue if the set temperature itself is high. It is assumed here to be the set temperature +1° C.

If the measured temperature does not exceed the upper limit (S23: N), the control advances to the step S28. On the other hand, if the measured temperature exceeds the upper limit (S23: Y), the control unit 105 instructs the laser generator 3 to bring down the laser power to the minimum value PWR_MIN (S24), and measure the temperature with the temperature measuring unit 106 (S25) and waits until the measured temperature comes down below the set temperature (S26).

When the measured temperature comes down below the set temperature, the control unit 105 instructs the laser generator 3 to adjust the power to a value a predetermined adjusting value PWR_CTRL_{—}2 (e.g., 1 W) lower than the power used before the measured temperature exceeded the upper limit (S27), and return to the step S21. However, it shall not be lowered below the predetermined value minimum PWR_MIN. PWR_CTRL_{—}2 can be either the same or different value as PWR_CTRL_{—}1.

Next, the control unit 105 makes a judgment as to whether the measured temperature is below the lower limit or not (S28). The lower limit value is also an arbitrary value that changes with the set temperature similar to the upper value. It is assumed here, for example, to be the set temperature −1° C. If the measured temperature is not below the lower limit (S28: N), the control advances to the step S30. On the other hand, if the measured temperature is below the lower limit (S28: Y), the control unit 105 instructs the laser generator 3 to increase the power by the predetermined mount PWR_CTRL_{—}2 (S29). However, it shall not exceed the predetermined maximum value PWR_MAX. After that, the process returns to the step S21.

Next, the control unit 105 compares the measured temperature, T_n, with the set temperature (S30). If the measured temperature is higher than the set temperature (S30: Y), it goes to the step S31, and goes to the step S33 otherwise.

The control unit 105 calculates the change rate of measured temperature since the last measurement T_n−T_n−1 in the step S31 and, if the change rate of measured temperature T_n−Tn−1 is not negative (S31: N), instructs the laser generator 3 to lower the power by a predetermined adjustment value PWR_CTRL_{—}3 (e.g. 1 W) (S32). This process corresponds to the pattern B or C of FIG. 8(a). However, it shall not be lowered below the predetermined value minimum PWR_MIN. On the other hand, if it is negative (S31: Y), it returns to the step S21 without making any power adjustment. PWR_CTRL_{—}3 can be either the same or different value as PWR_CTRL_{—}1 and PWR_CTRL_{—}2.

Next, the control unit 105 compares the measured temperature, T_n, with the set temperature (S33). If the measured temperature is lower (S33: Y), it advances to the step S34. Otherwise, it returns to the step S21 (corresponds to FIG. 8(b)).

The control unit 105 calculates the change rate of measured temperature since the last measurement T_n−T_n−1 in the step S34 and, if the change rate of measured temperature T_n−Tn−1 is not positive (S34: N), instructs the laser generator 3 to raise the power by a predetermined adjustment value PWR_CTRL_{—}3 (S35). This process corresponds to the pattern A or B of FIG. 8(c). However, it shall not be raised above the predetermined value maximum PWR_MAX. On the other hand, if it is positive (S34: Y), it returns to the step S21 without making any power adjustment.

Next, an example of temperature control performed according to the above process sequence will be described.

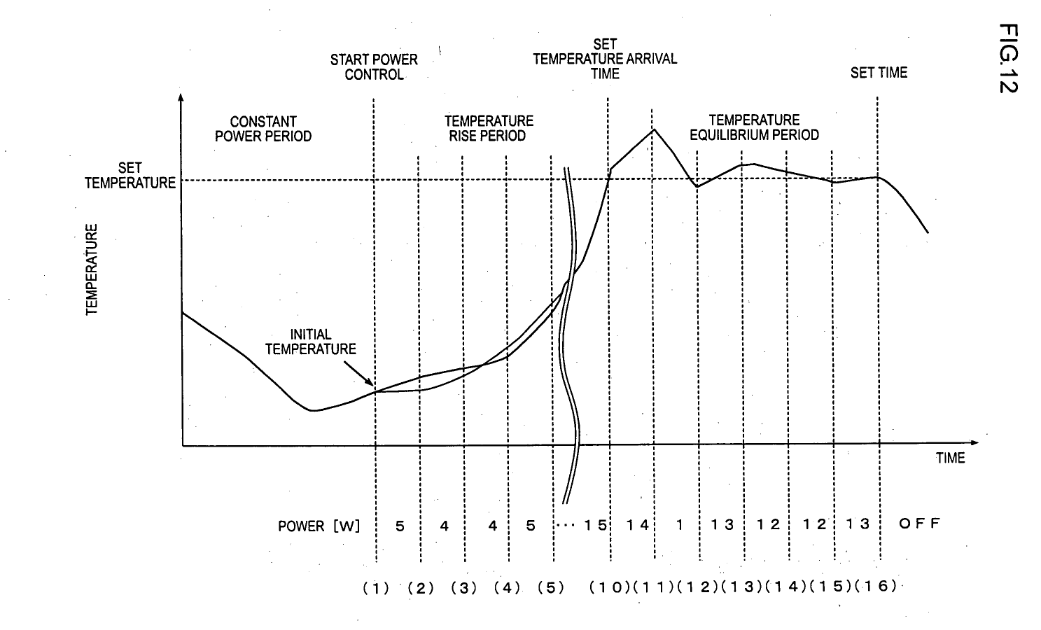

FIG. 12 is a diagram showing an example of temperature control. In the following description, the “S numbers” correspond to those S numbers used in the above description of the process procedure respectively, so that only those steps required for the temperature control example will be described below avoiding duplicate descriptions. Also, in the following descriptions, (1), (2), . . . indicate that they correspond to the processes described with reference to (1), (2), . . . in FIG. 12.

First, a relatively low 5 W constant laser power is applied. It is possible that we see uncertain temperature performances in accordance with body tissue temperature and cooling water conditions, etc.

Once the constant power time is over in (1), the initial temperature will be measured and the temperature rise curve will be calculated (S3).

In (2), since the measured temperature (solid line) is higher than the temperature which is supposed to be at present time (phantom line) CT_n, and the change rate of measured temperature since the last measurement T_n−T_n−1 is larger than the change rate required to change from the present measured temperature to the calculated temperature of the next target CT_n+1−T_n, the power value is lowered by 1 W to 4 W (S10).

In (3), although the measured temperature (solid line) is higher than the temperature which is supposed to be at present time CT_n, the change rate of measured temperature since the last measurement T_n−T_n−1 is smaller than the change rate required to change from the present measured temperature to the calculated temperature of the next target CT_n+1−T_n, so that the power value is not changed (S9: N).

In (4), since the measured temperature is lower than the temperature which is supposed to be at present time CT_n and the change rate of measured temperature since the last measurement T_n−T_n−1 is smaller than the change rate required to change from the present measured temperature to the calculated temperature of the next target CT_n+1−T_n, the power value is raised by 1 W to 5 W (S13).

In (5) and thereafter, the control is executed in accordance with the process sequence described before.

In (10), since the measured temperature exceeded the set temperature, it shifts to the temperature equilibrium period control (S6: Y). Since the measured temperature is higher than the set temperature and the change rate of measured temperature T_n−T_n−1 is not negative, the power value is lowered by 1 W to 14 W (S32).

In (11), since the measured temperature exceeded the upper limit, the power value is set to the minimum value 1 W (S24).

In (12), since the measured temperature became lower than the set temperature, the power value is set to 1 W lower than the power value used before the upper limit was exceeded, i.e., to 13 W (S27).

In (13), since the measured temperature is higher than the set temperature and the change rate of measured temperature T_n−T_n−1 is not negative, the power value is lowered by 1 W to 12 W (S32).

In (14), although the measured temperature is higher than the set temperature, the change rate of measured temperature T_n−T_n−1 is negative, so that the power value is not changed (S31: Y).

In (15), since the measured temperature is lower than the set temperature and the change rate of measured temperature T_n−T_n−1 is not positive, the power value is raised by 1 W to 13 W (S35).

In (16), the laser is turned off since the set time has passed (S36).

As described in the above, the present embodiment makes it possible to maintain the heating range constant and stabilize it in the target area where treatment is required by changing the heating temperature in accordance with the predetermined target temperature change pattern (see detail in the embodiment). Also it is possible to raise temperature in a stable manner along the temperature change pattern of the target temperature change pattern (especially temperature rise curve) with the laser power control mentioned above.

### Second Embodiment

The second embodiment is to modify the period setting of the constant power period in the first embodiment from ending the period by a set time to ending the period when the temperature change turns upward. Other elements of the constitution such as the structure of the apparatus and process sequence other than the constant power period remain the same as in the first embodiment.

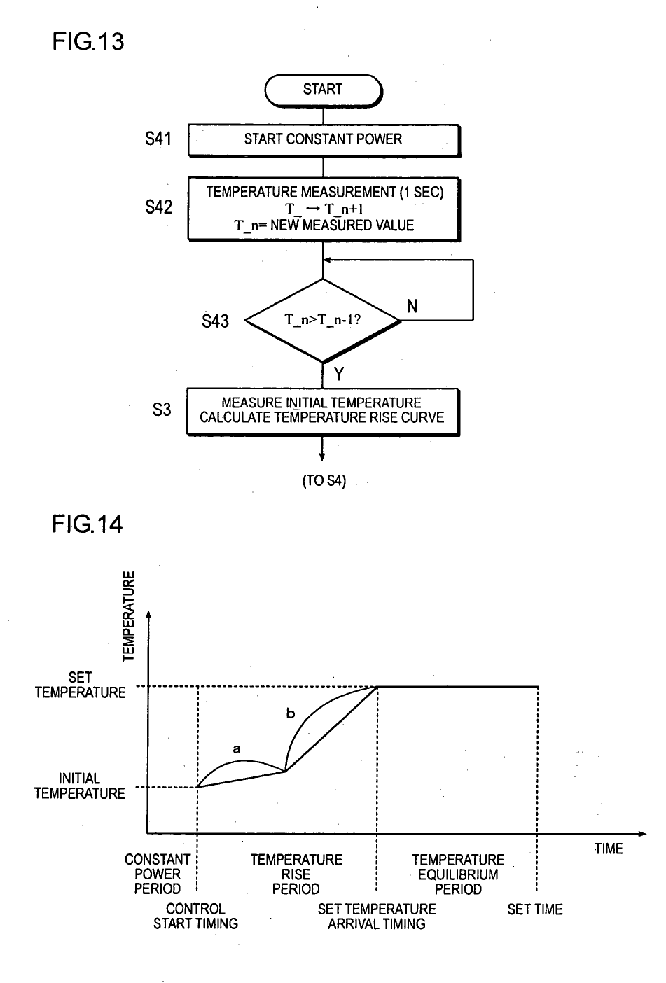

FIG. 13 is a flow chart showing the temperature control process sequence of the second embodiment. Since the processes other than those described bellow are identical to those processes for temperature control already described in the first embodiment, their descriptions are not repeated here.

The control unit 105 instructs the laser generator 3 to maintain a predetermined constant power value when the treatment is started (S41). The power value at this time is, for example, 5 W.

Next, the control unit 105 instructs the temperature measuring unit 106 to measure the current temperature and obtains the temperature measurement result (S42). The measurement interval is, for example, 1 second. The value of the last measurement will be stored as T_n−1 and the value of the new measurement as T_n.

Next, the control unit 105 continues to maintain the same value until the measured temperature becomes higher than the last measured value (S43), and advances to the step S3 (same as FIG. 10) when the temperature starts to go up.

From then on, the step S4, which was described in the first embodiment, and the processes thereafter (see FIGS. 10 and 11) shall follow.

As can be seen from the above, the second embodiment increases the possibility of advancing to the temperature rise period faster as the termination of the initial constant power period is detected by whether a specified temperature has been reached or not rather than a specified time has elapsed. Therefore, it is possible to shorten the total body tissue irradiation time. This judgment during the constant power period based on the temperature measurement result can be so constituted as to judge the termination of the constant power period in accordance with the change rate of the temperature measurement (for example, to detect the timing when the temperature change becomes zero, i.e., the temperature change has become constant, or the temperature rise exceeds a predetermined rate).

### Third Embodiment

The third embodiment is different from the first and second embodiments in that the temperature rise curve in the temperature rise period is defined by a plurality of mathematical formulas.

Therefore, the third embodiment is different from the first and second embodiments only in the formulas that are used in calculating the temperature rise curve in the step S3 and the rest of the embodiment including the constitution of the apparatus and the process sequence is identical to those of the other emobodiments. Therefore, the descriptions of those similar features are not repeated here.

FIG. 14 is a graph showing the target temperature change pattern in the third embodiment.

As can be seen from the drawing, the temperature rise curve of the temperature rise period is expressed by two line segments, zone-a and zone-b, in the third embodiment.

Therefore, the formulas for this period are:
in zone-a, temperature T_{—}a=a×time+initial temperature (3)
in zone-b, temperature T_{—}b=b×time+T_{—}a (4)
where a and b are coefficients for determining the temperature rise rate for the zone-a and the zone-b.

Thus, it is possible to reduce the amount of calculation necessary for setting the target temperature during the control as the temperature rise curve is assimilated by a plurality of line segments in the third embodiment.

### Fourth Embodiment

The fourth embodiment is to perform the power control in the constant power period by means of measuring the temperature of the cooling water circulating through the applicator 1.

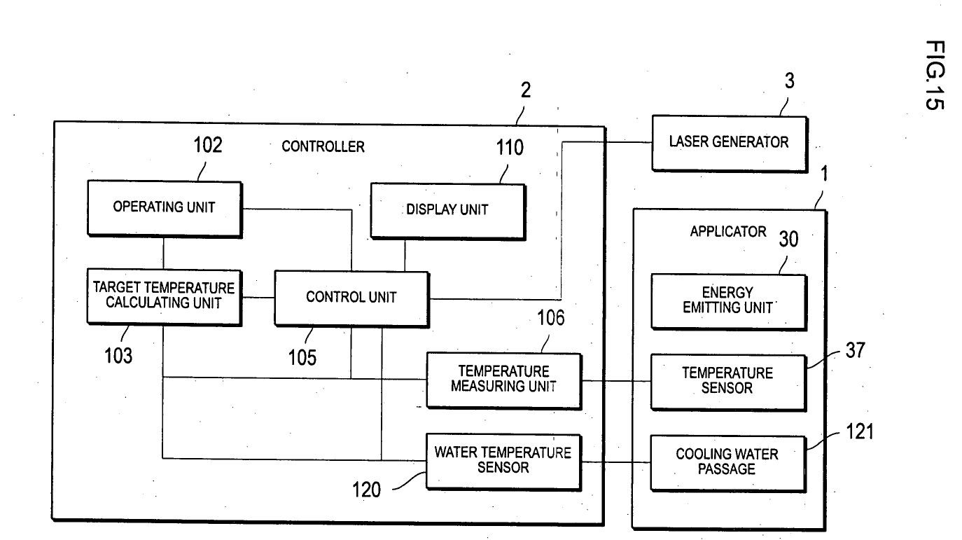

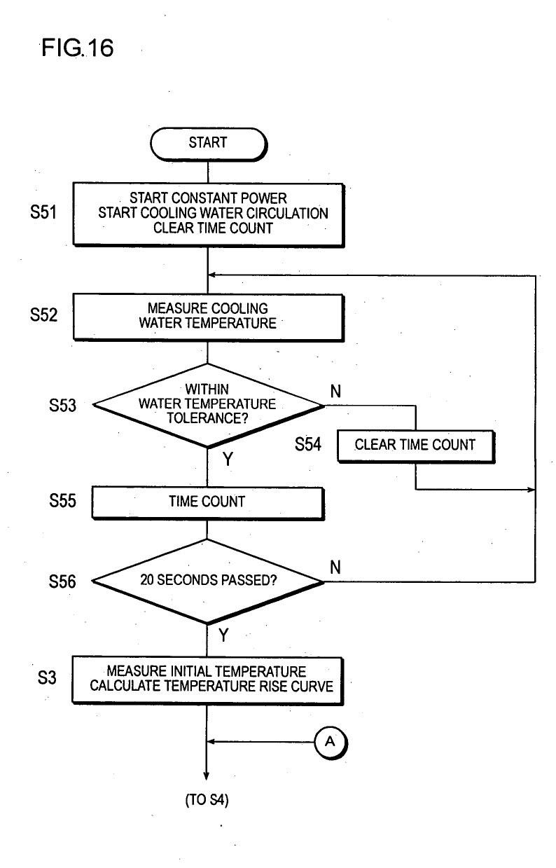

FIG. 15 is a functional block diagram of the controller 2 in the fourth embodiment, and FIG. 16 is a flowchart for showing the process sequence of the temperature control in the fourth embodiment.

The controller 2 in the fourth embodiment has a cooling water passage 121 provided in the applicator 1. The cooling water passage 121 contains the supply passage 91 and the drain passage 92. The controller 2 has a water temperature sensor 120 for measuring the temperature of the cooling water that flows through the cooling water passage 121. Other constitutions of the present embodiment are identical to those of the first embodiment so that their descriptions are not repeated here.

The water temperature sensor 120 is a liquid temperature measuring unit. The water temperature sensor 120 measures the temperature of the cooling water in the cooling device. The water temperature sensor 120 is preferably provided at a location where the temperature of the water returning from the applicator 1 can be measured. It is so that it is possible to measure the temperature of the cooling water after it absorbing the heat in the applicator 1 as it passes through the applicator 1 by measuring the water temperature in the return passage from the application 1.

In the fourth embodiment, the control unit 105 obtains the cooling water temperature measured by the water temperature sensor 120, and determines the termination of the constant power period from said temperature.

The temperature control will be described with reference to the flowchart shown in FIG. 16.

The control unit 105 instructs the laser generator 3 to maintain a predetermined constant power value when the treatment is started (S51). The power value at this time is, for example, 5 W. The control unit 105 simultaneously instructs the cooling device (not shown) to start the pump for circulating the cooling water. Also, it clears the time count.

The control unit 105 then obtains the current cooling water temperature from the water temperature sensor 120 (S52).

The control unit 105 then makes a judgment whether the cooling water is within the tolerance range (S53). The tolerance range means a set temperature during the constant power period, for example, 22±1° C. If the cooling water temperature is outside of the tolerance range (S53: N), it clears the time count and returns to the step S52 (S54). On the other hand, if the cooling water temperature is within the tolerance range (S53: Y), it counts the time the temperature is maintained within the tolerance range (S55).

Following the step S55, the control unit 105 makes a judgment whether the time the temperature is maintained within the tolerance range has passed 20 seconds (S56). If it has not passed 20 seconds (S56: N), it returns to the step S52. In other words, the laser power is maintained constant during this period.

On the other hand, if it has passed 20 seconds (S56: Y), it judges that enough time has passed maintaining the temperature within the tolerance range measures the initial temperature, calculates the temperature increase curve, and starts the power control (S3 (same as shown in FIG. 10)).

The control unit 105 then performs the step S4, which was described in the first embodiment, and the processes thereafter (see FIGS. 10 and 11). It is also preferable to constitute the system in such a way as to turn off the laser in the step S14 and turn off the pump to stop the circulation of the cooling water simultaneously, when the set time has passed in the step S4 (S4: Y).

As can be seen from the above, the fourth embodiment makes it possible to detect temperature stabilization without being affected by temperature increase of the body (e.g., urethra) where the applicator 1 is inserted by measuring the cooling water temperature and detecting a stabilized status of the cooling water temperature in order to make a judgment that the constant power period is over when the cooling water has been stabilized for a predetermined time.

Although the cooling water circulation is controlled by turning on the pump with the start of the laser irradiation and turning off the pump with the end of the laser irradiation in this fourth embodiment, it is also possible to constitute in such a way as to turning on the pump before starting the laser irradiation and turning off the pump after the laser irradiation is terminated.

### EXAMPLE

A practical example of the invention will be described below. This example is a test result of measuring the change of body tissue temperature when a body tissue sample was irradiated by laser according to various temperature rise curves.

First, let us describe the constitution of a deep area temperature sensor used in this example for measuring the temperature of deep area body tissue.

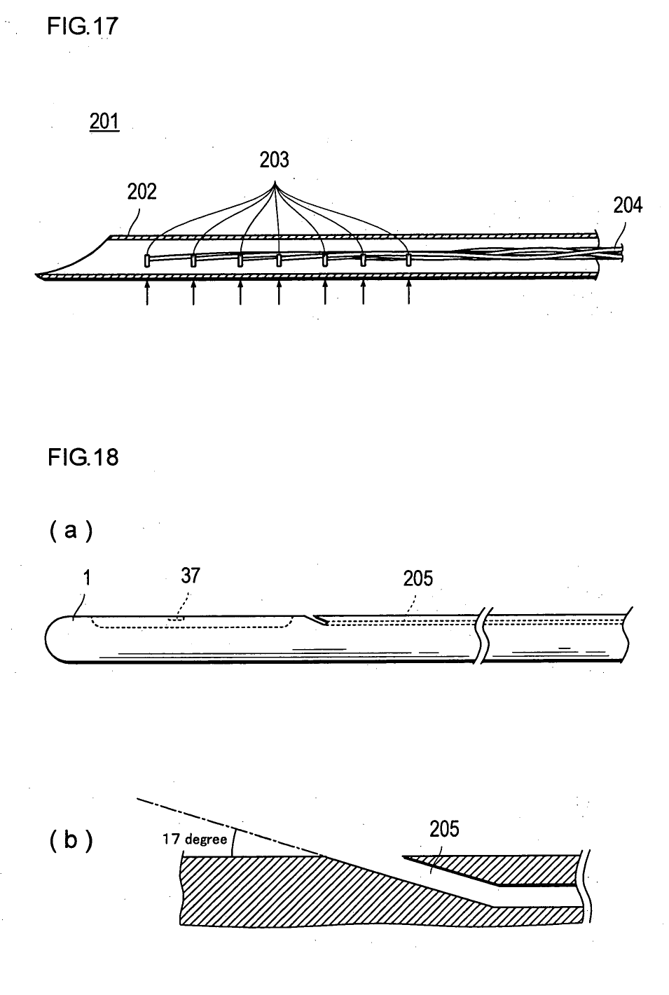

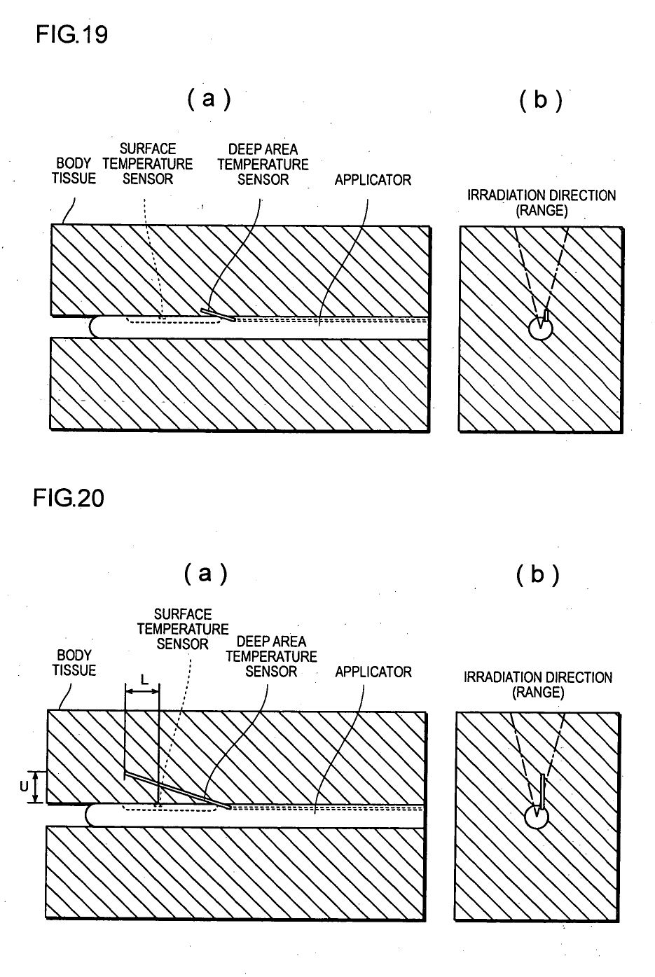

FIG. 17 is a cross section showing the constitution of the deep area temperature sensor used for measuring the temperature of deep areas of body tissue. FIG. 18 is an outline drawing of an applicator equipped with a special lumen for mounting the deep area temperature sensor consisting of a side view (a) and an enlarged view of the part from which the deep area temperature sensor protrudes. FIG. 19 and FIG. 20 are cross sections showing the applicator and the deep area temperature sensor being inserted into body tissue (in both FIG. 19 and FIG. 20, a drawing (a) represents a side cross section of the applicator 1 and a drawing (b) represents a cross section perpendicular to the applicator's insertion direction).

The deep area temperature sensor 201 shown in FIG. 17 has a diameter of 0.7 to 1 mm, a plurality of thermocouples 203 (seven pieces in this case) provided with an interval of 3 mm (as shown by an arrow in the drawing) in a steel pipe 202 (e.g., stainless pipe) which is machined to form a needle-like shape at the distal end, and a lead wire 204 running through the metal pipe 202 from each thermocouple 203 to the outside. The lead wire 204 connects to a temperature measuring device (not shown), which calculates the temperature at each thermocouple position based on the electrical signal from each thermocouple 203. Thus, the temperature at each thermocouple position is measured.

As shown in FIG. 18, in order to thread this deep area temperature sensor 201 through the applicator 1, a special lumen 205 for the temperature sensor is provided in the applicator 1 by brazing or other similar methods. The distal end of the lumen for the temperature sensor is formed in a specific angle (e.g., 170) extending outward of the applicator 1.

The deep area temperature sensor 201 is installed as shown in FIG. 19 by first inserting the applicator 1 into the human body and locating it in a position appropriate for laser irradiation, puncturing the human body with the deep area temperature sensor 201 through the temperature sensor lumen 205, and inserting the deep area temperature sensor 201 further deeper into the tissue as shown in FIG. 20.

The deep area temperature sensor 201 efficiently enters into the inside of the human body because of the angle provided at the distal end of the temperature sensor lumen 205.

In order to make sure that the deep area temperature sensor 201 is almost fully stuck in the human body, it is so designed in this example that the depth from the outer surface of the applicator that the tip of the sensor reaches U be 10 mm, and the distance of said sensor from the surface temperature sensor 37 L be 12 mm.

Such a deep area temperature sensor 201 is used for measuring the deep area of the human body and is not necessarily needed for a normal hyperthermia treatment.

A body tissue sample that has substantially similar laser absorbency and diffusion characteristic as body tissue was used as the body tissue sample in this example. Albumin Phantom, for example, can be used as such a body tissue sample. This is a type of agar added with pigment called naphthol green and chicken egg albumin. According to a paper “Lasers in Surgery and Medicine,” 25: 159-169 (1999) by M. N. Iizuka, such a sample can be adjusted to assimilate with body tissue's laser absorbancy and diffusion characteristic by controlling the amounts of naphthol green and chicken egg albumin to be added.

In this example, albumin phantom was prepared as the body tissue sample with a composition of water 88.7 (mass %), agar powder 1.4 (mass %), chicken egg albumin 5.8 (mass %), and 0.0387 vol. % naphthol green water solution 4.1 (mass %).

In this example, the experiment was conducted by inserting the applicator 1 into the body tissue sample and affixing the laser irradiation part. The target temperature pattern was set to assimilate FIGS. 5(a) and (b) conditions, controlling the laser power according to the first embodiment. The initial temperature and the set temperature were also arranged to be the same. The puncturing position of the deep area temperature sensor 201 into the body tissue sample was as described above.

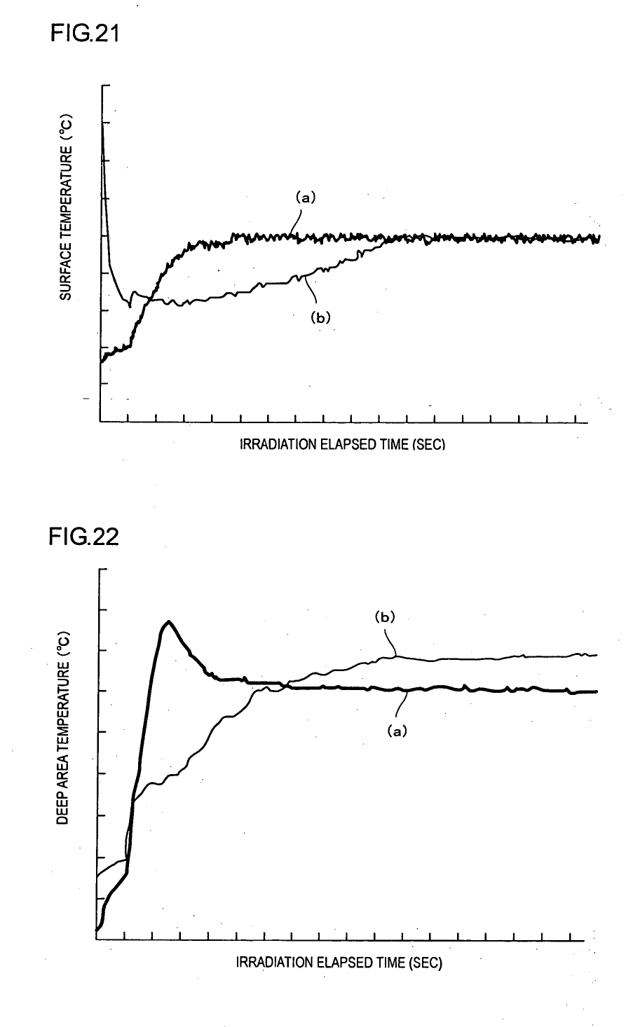

FIG. 21 is a graph showing the surface temperature of the body tissue sample relative to the laser irradiation time measured by the temperature sensor 37. FIG. 22 is a graph showing the temperature measurement data of the body deep area relative to the laser irradiation time. What is shown here is the temperature measured by the thermocouple located at the fifth position from the proximal end of the seven thermocouples 203 shown in FIG. 17.

As can be seen from FIG. 21, the body tissue surface temperatures went through changes in the similar ways as their respective target temperature change patterns.

In contrast to that, the measurement data graph of the deep area temperature sensor 201 shown in FIG. 22 indicates that the temperature rises sharply in the initial stage of irradiation in case of (a) in a deep area of the body tissue sample, which is followed by a temperature drop, and then reaches equilibrium at a certain temperature. On the other hand, in case of (b), the temperature rises gradually in the initial stage of irradiation and reaches equilibrium at a level higher than that of (a).

After the laser irradiation, the body tissue sample was sliced in such a way that a knife goes through the area where the surface temperature sensor of the applicator was approximately located to check the range of thermal degeneration in this example.

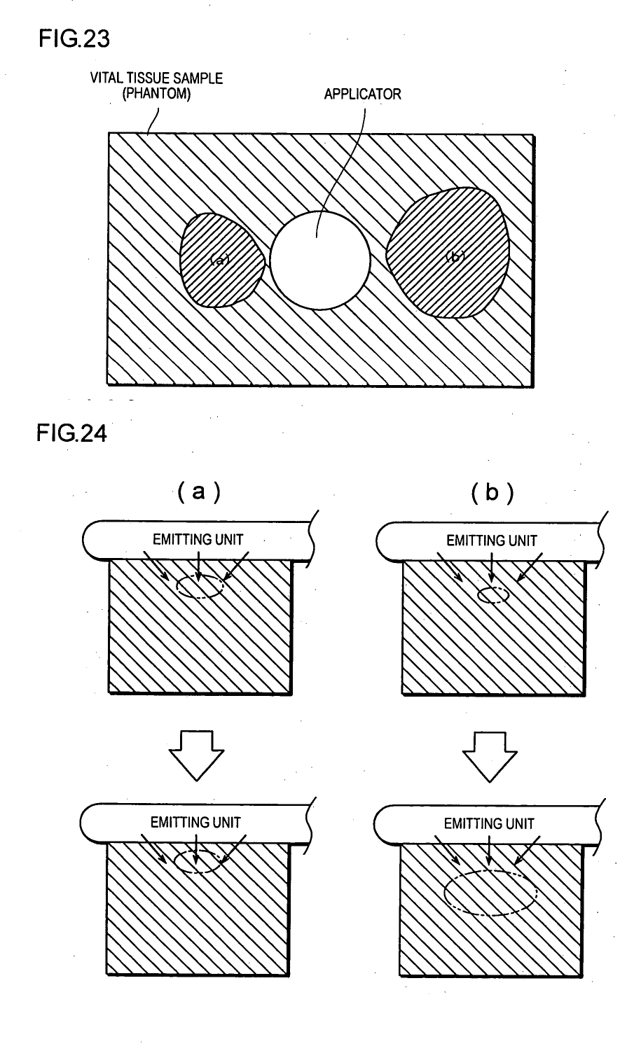

FIG. 23 is a model cross-sectional diagram of a body tissue sample after laser irradiation.

As can be seen from this diagram, the thermally degenerated range of the body tissue sample is larger in case of the target temperature change pattern (b) and less likely to heat the surface layer vicinity than in case of (a). In other words, by setting up a target temperature change pattern which allows the differential coefficient obtained by differentiating temperature with time to increase with time (see the formula (2) described before) and controlling the temperature by monitoring the body tissue surface temperature including the targeted heating area to follow this temperature change pattern during the heating period, it is possible to heat only deep areas without causing any thermal degeneration or coagulation in the body tissue surface layers.

FIG. 24 shows diagrams for describing thermal degeneration of body tissue due to laser irradiation. Diagrams (a) and (b) correspond to the target temperature change patterns (a) and (b) respectively.

The major reason why such differences occur is that laser absorption and diffusion intensities increase with heat degeneration and coagulations of body tissue and the highest temperature point moves toward the surface layer. More specifically, the pattern (a), which provides faster temperature rise, tends to cause heat degeneration in the highest temperature point in an earlier period of irradiation and cause it to move toward the surface layer. On the other hand, the pattern (b), which provides slower temperature rise, tends to cause heat degeneration in the highest temperature point less likely to move toward the surface layer compared to the pattern (a).

Thus, the target temperature change pattern (b) makes it easier to maintain the laser's deep-reaching capability in body tissue deep areas and to achieve a wider area of heating within the same period of heating time, so that it is easier to achieve wider areas of heat degeneration and coagulation. On the contrary, if a heat degeneration pattern is set such as in (a), it is also possible to heat a smaller area, thus limiting heat generation and coagulation areas.

As can be seen from the above descriptions and the present example, the present invention makes it possible to set up an arbitrary target temperature change pattern to control the range of hyperthermia treatment of living tissue arbitrarily.

The embodiments and example described above shall not be construed to limit the present invention and various variations are possible by a person skilled in the art all of which belong to the present invention.

For example, energy to be used in the present invention is not limited to laser but includes microwaves, RF waves, HF waves, ultrasonic waves and any other that can heat body tissue, and they can be controlled in manners similar to those described in the above embodiments and example. The target temperature change pattern is not limited to a combination of primary or quadratic equations.

The apparatus for hyperthermia treatment of the present invention can be applied to various hyperthermia treatments of living tissue, for example, it can be applied to a hyperthermia treatment of prostatic diseases such as benign prostatic hyperplasia and prostate cancer without causing any heat degeneration in normal tissue of urethra and rectum, while applying treatment only to the interior of the prostate gland. It can also be applied not only to prostate gland but also to any other diseases where it is desired to heat only deep body tissue while maintaining body tissue surface layers intact.

This application is based on Japanese Patent Application No. 2004-104978 filed on Mar. 31, 2004, the contents of which are hereby incorporated by reference.

1. An apparatus for hyperthermia treatment comprising:
a heating unit configured to heat a target area of body tissue;
a temperature measuring unit configured to measure a tempereture of said body tissue including said target area;
a target temperature setting unit operatable to set a temperature change pattern for said heating unit; and
a control unit operatable to control said heating unit in such a manner that the temperature measured by said temperature measuring unit change in accordance with said temperature change pattern.

2. An apparatus for hyperthermia treatment as claimed in claim 1 further comprising:
a temperature change rate calculation unit operatable to calculate chronological change rates of measured temperatures measured by said temperature measuring unit; and
a target change rate calculation unit operatable to calculate necessary chronological temperature change rates based on comparisons of said measured temperature with said temperature change pattern set by the target temperature setting unit, wherein
said control unit controls said heating unit in such a manner as to follow said temperature change pattern based on discrepancies between said temperature change pattern and the measured temperature measured by said temperature measuring unit, the chronological change rates of the measured temperature calculated by said temperature change rate calculation unit, and the necessary chronological temperature change rates calculated by said target change rate calculation unit.

3. An apparatus for hyperthermia treatment as claimed in claim 1, wherein the temperature change pattern that is set as such that their differential coefficient, which is obtained by differentiating temperature with time, increase with time during a period of heating by said heating unit.

4. An apparatus for hyperthermia treatment as claimed in claim 1, wherein said temperature change pattern has a plurality of change rates.

5. An apparatus for hyperthermia treatment as claimed in claim 1, wherein said heating unit comprises:
an energy generating unit for generating energy for heating said body tissue;
an emitting unit configured to emit said body tissue with the energy generated by said energy generating unit;
a drive unit for moving an energy emitting position of said emitting unit within a predetermined range; and
an angle changing unit for changing said energy emitting angle of said emitting unit in accordance with motion of said emitting position so that the energy emitted by said emitting unit concentrate on a predetermined target area.

6. An apparatus for hyperthermia treatment as claimed in claim 5, wherein said energy is laser.

7. An apparatus for hyperthermia treatment as claimed in claim 1, wherein said emitting unit comprises:
a flow passage for fluid to flow through said emitting unit,
said flow passage is connected to a fluid supply unit for supplying said fluid.

8. An apparatus for hyperthermia treatment as claimed in claim 7, wherein said fluid supply unit causes said fluid to circulate through said fluid passage.

9. An apparatus for hyperthermia treatment as claimed in claim 1, wherein
said control unit maintains said heating unit's heating power constant for a period from said heating unit's start of heating until a predetermined condition is met.

10. An apparatus for hyperthermia treatment as claimed in claim 9, wherein
said condition is a predetermined time elapsed from said heating unit's start of heating.

11. An apparatus for hyperthermia treatment as claimed in claim 9, wherein
said condition is either a temperature value measured by said temperature measuring unit or a change rate of the measured value of temperature.

12. An apparatus for hyperthermia treatment as claimed in claim 7 further comprising:
a fluid temperature measuring unit for measuring said fluid's temperature,
said control unit maintains said heating unit's heating power constant until the measured value of said fluid temperature measuring unit meets a predetermined condition.

13. A method for hyperthermia treatment comprising:
setting a temperature change pattern;
measuring a temperature of a body tissue including a target area; and
heating the target area so that the measured temperature changes in accordance with said temperature change pattern.

14. A method for hyperthermia treatment as claimed in claim 13 further comprising:
calculating a chronological change rate of the measured temperature;
calculating a necessary chronological change rate based on comparison of the measured temperature with said temperature change pattern;
determining a power of a heating unit based on discrepancy between said temperature change pattern and the measured temperature, said chronological change rate and said necessary chronological change rate; and
controlling said heating unit with the determined power.

15. A method for hyperthermia treatment as claimed in claim 14, wherein
controlling said heating unit with a constant power until a predetermined condition is met.

16. A method for hyperthermia treatment as claimed in claim 15 further comprising:
supplying fluid for cooling the body tissue.

17. A method for hyperthermia treatment as claimed in claim 15, wherein
said condition is a predetermined time elapsed from said heating unit's start of heating.

18. A method for hyperthermia treatment as claimed in claim 15, wherein
said condition is either the measured temperature or the calculated chronological change rate.

19. A method for hyperthermia treatment as claimed in claim 16, wherein
said condition is that a temperature of the fluid meets a predetermined value.
